(19) United States
(12) Patent Application Publication
(10) Pub. No.: US 2018/0338910 A1
(43) Pub. Date: Nov. 29, 2018

(54) INTRANASAL ADMINISTRATION OF GLUTAMATE CARBOXYPEPTIDASE (GCP-II) INHIBITORS

(71) Applicant: THE JOHNS HOPKINS UNIVERSITY, BALTIMORE, MD (US)

(72) Inventors: BARBARA S. SLUSHER, KINGSVILLE, MD (US); RANA RAIS, OWINGS MILLS, MD (US)

(73) Assignee: THE JOHNS HOPKINS UNIVERSITY, BALTIMORE, MD (US)

(21) Appl. No.: 15/542,175

(22) PCT Filed: Jan. 11, 2016
(86) PCT No.: PCT/US16/12856

§ 371 Date: Jul. 7, 2017

Related U.S. Application Data
(60) Provisional application No. 62/101,437, filed on Jan. 9, 2015.

(51) Int. Cl. A61K 9/00; A61K 31/662; A61P 25/28; A61K 49/00; A61K 51/04; A61K 31/194; A61K 31/27

(52) U.S. Cl. CPC A61K 9/0043; A61K 31/662; A61P 25/28; A61K 31/27; A61K 51/0489; A61K 31/194; A61K 49/0052

(57) ABSTRACT

The presently disclosed subject matter provides methods for treating and diagnosing neurological diseases or disorders using intranasal administration of glutamate carboxypeptidase II (GCP-II) inhibitors in a subject. Methods for imaging GCP-II in a subject, including imaging of the brain and/or peripheral nervous system, also are provided.

### CROSS-REFERENCE TO RELATED APPLICATIONS

This application claims the benefit of U.S. Provisional Application No. 62/101,437, filed Jan. 9, 2015, which is incorporated herein by reference in its entirety.

### FEDERALLY SPONSORED RESEARCH OR DEVELOPMENT

This invention was made with government support under R01 CA61056 awarded by the National Institutes of Health (NIH). The government has certain rights in the invention.

### BACKGROUND

Elevated levels of glutamate, a major neurotransmitter in the central and peripheral nervous system, is often associated with excitotoxicity, which is a hallmark of many neurological and psychiatric disorders (Mesters et al., 2006; Watkins, 2000; Carpenter and Dickenson, 2001). One strategy to reduce the levels of extracellular glutamate involves the inhibition of the brain enzyme glutamate carboxypeptidase II (GCP-II) (EC3.4.12.21), a membrane-bound zinc metalloprotease involved in the hydrolysis of the abundant neuropeptide N-acetylaspartylglutamate (NAAG) to N-acetylaspartate (NAA) and L-glutamate (Mesters et al., 2006; Slusher et al., 1999; Barinka et al., 2012). NAAG is released from neurons/axons after depolarization (Neale et al., 2000) and acts as an agonist at presynaptic metabotropic glutamate 3 receptors (mGluR3) (Olszewski et al., 2012) which limits further glutamate release, although controversy exists around this finding (Chopra et al., 2009; Neale, et al., 2011). Released NAAG can also be catabolized by GCP-II, liberating glutamate, which can serve as an agonist at various glutamate receptors. Inhibition of GCP-II results in both increased extracellular NAAG and decreased extracellular glutamate. Both of these effects dampen glutamate transmission and can afford neuroprotection.

In support of this, small molecule inhibitors of GCP-II have been demonstrated to be efficacious in multiple preclinical models wherein excess glutamate transmission is implicated including traumatic spinal cord and brain injury (Long et al., 2005; Zhong et al. 2005; Zhong et al. 2006) stroke (Slusher et al., 1999), neuropathic and inflammatory pain (Jackson et al. 2001; Kozikowski et al. 2004; Chen, et al., 2002; Yamamoto et al., 2001; hang et al., 2006; Yamamoto et al. 2004; Yamamoto et al. 2007; Adedoyin et al., 2010; Carpenter, et al., 2003; Nagel et al. 2006; Sasson et al., 2008; Yamamoto et al., 2008; Zhang et al., 2002; Saito et al., 2006), ALS (Ghadge, et al., 2003), schizophrenia (Olszewski et al., 2004), neuropathy (Berent-Spillson et al., 2004; Carozzi et al., 2010), drug abuse (McKinzie et al., 2000; Shinppenberg et al., 2000; Xi et al., 2010) and cognition (Rahn et al., 2012). In addition, GCP-II knockout animals have shown to be protected against ischemic brain injury, peripheral neuropathy (Bacich et al., 2005), and have demonstrated long term memory enhancing effects (Janczura et al., 2013).

Several GCP-II inhibitors with different chemical scaffolds have been synthesized over the last two decades including those with phosphonate (e.g. 2-(phosphonomethyl)-pentanedioic acid, 2-5 PMPA), thiol (e.g., 2-(3-mercaptopropyl)pentane-dioic acid; 2-MPPA) and urea moieties (e.g. (N—[N—[(S)-1,3-dicarboxypropyl]carbamoyl]-L-cysteine; DCMC) (Barinka et al., 2012). Potent GCP-II inhibitors identified to date have required two functionalities—a glutarate moiety that binds the C-terminal glutamate recognition site of GCP-II, and a zinc chelating group to engage the divalent zinc atoms at the enzyme's active site (Barinka et al., 2012). Although inclusion of these functionalities has led to highly potent inhibitors, the compounds suffer from being exceedingly hydrophilic and show low membrane permeability. The only GCP-II inhibitor class to show oral bioavailability was the thiol-based inhibitors, with 2-MPPA advancing into clinical studies (Van der Post et al., 2005). Unfortunately, subsequent immunological toxicities (common to thiol drugs) were observed in primate studies which halted its development.

The phosphonate based inhibitor 2-(phosphonomethyl)-pentanedioic acid (2-PMPA) is extremely potent (IC₅₀=300 pM), selective (Slusher et al., 1999; Jackson et al., 2001), and has demonstrated therapeutic benefit in over twenty in vivo models of neurological disorders performed by several independent laboratories (Slusher et al., 1999; Chen, et al., 2002; Yamamoto et al., 2001; Tortella et al., 2000; Wozniak et al., 2012; Witking et al., 2002; Luszczki et al., 2006; Popik et al., 2003). Despite its picomolar potency, most preclinical studies have administered 2-PMPA at doses of 50-100 mg/kg intraperitoneal (i.p.) or intravenous (i.v.) to produce therapeutic effects, as the compound is highly hydrophilic and has limited oral bioavailability and tissue penetration (Rais et al., 2014). Similar limitations have been met with urea-based inhibitors, which have mainly been utilized as peripheral imaging agents (Foss et al., 2005).

Therefore, the only routes of administration employed to date for successful delivery of GCP-II inhibitors are very high doses via parenteral routes (due to limited oral bioavailability) and the intrathecal route which is invasive in nature, with the exception of the thiol-base inhibitors which were found to be orally bioavailable but not brain penetrable. The pressing need to move these efficacious, but hydrophilic compounds into the clinic emphasizes the significance of exploring alternative patient compliant routes of administration.

### SUMMARY

In one aspect, the presently disclosed subject matter provides a method for systemic delivery of a glutamate carboxypeptidase II (GCP-II) inhibitor to a subject via an intranasal route, including delivery to the brain and/or peripheral nervous system. In particular aspects, the GCP-II inhibitor is urea, hydroxamate, thiol, or phosphonate based, including, but not limited to, (N—[N—[(S)-1,3-dicarboxypropyl]carbamoyl]-L-cysteine) (DCMC), 2-(3-mercaptopropyl)pentane-dioic acid (2-MPPA) or 2-(phosphonomethyl)-pentanedioic acid (2-PMPA), and stereoisomers and prodrugs thereof.

In other aspects, the presently disclosed subject matter provides a method for treating a neurological disease or disorder in a subject in need of treatment thereof, the method comprising intranasally administering to the subject a therapeutically effective amount of glutamate carboxypeptidase II (GCP-II) inhibitor. In particular aspects, the neurological disease or disorder is selected from the group consisting of traumatic spinal cord and brain injury, stroke, neuropathic and inflammatory pain, neurological disorder as a result of drug abuse, epilepsy, amyotrophic lateral sclerosis (ALS), schizophrenia, Huntington's disease, neuropathy, multiple sclerosis, cognition impairment, brain cancer, HIV-associated neurocognitive disorder, and cognition impairment associated with neurodegenerative or neuropsychiatric conditions.

In some aspects, the presently disclosed subject matter provides a method for diagnosing a neurological disease or disorder involving alteration of glutamate carboxypeptidase II enzyme (GCP-II) levels or location in the brain and/or peripheral nervous system of a subject, the method comprising intranasally administering to the subject an effective amount of GCP-II inhibitor labeled with a fluorescent species or radiolabeled with an isotope and obtaining an image of the brain of the subject, wherein an alteration in levels or location of GCP-II in the brain and/or peripheral nervous system as compared to the brain and/or peripheral nervous system of a subject without the neurological disease or disorder is indicative that the subject has the neurological disease or disorder.

In certain aspects, the presently disclosed subject matter provides a method for systemically imaging glutamate carboxypeptidase II (GCP-II), including imaging in the brain and/or peripheral nervous system of a subject, the method comprising intranasally administering to the subject an effective amount of GCP-II inhibitor labeled with a fluorescent species or radiolabeled with an isotope and obtaining an image of the brain and/or peripheral nervous system, or other organ or system of interest, of the subject.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows the mean concentrations of 2-PMPA, 2-MPPA and DCMC in different brain regions. The concentrations were measured in olfactory bulb, cortex and cerebellum following 30 mg/kg intranasal administration in rats. The tissues were collected 1 h post dose and evaluated via LC/MS/MS;

FIG. 2A and FIG. 2B show the mean concentration in ng/mL or ng/g versus the time profiles in hours for 2-PMPA in rat plasma, olfactory bulb, cortex and cerebellum following: (FIG. 2A) 30 mg/kg intraperitoneal; and (FIG. 2B) 30 mg/kg intranasal administration;

FIG. 3 shows a brain tissue to plasma (B/P) ratio of 2-PMPA in different brain regions. The B/P ratio was calculated based on area under the curve (AUC₀₋ₜ) following 30 mg/kg intranasal or intraperitoneal administration;

FIG. 4 shows the ex vivo GCP-II enzymatic activity following 2-PMPA intranasal administration. The enzyme activity was measured in olfactory bulb, cortex and cerebellum collected 1 h post dose following 30 mg/kg intranasal administration. The percent inhibition was calculated in all tissue samples relative to brain tissues collected from untreated control rats;

FIG. 5 is a synthesis scheme for the presently disclosed derivatized 2-PMPA bioanalysis. The reaction was carried out using n-butanol with 3N HCl at 60° C. for 30 minutes, leading to formation of n-butyl esters of 2-PMPA carboxylic acids; and

FIG. 6 shows the brain exposure of i.n. administered R-2-PMPA versus S-2-PMPA.

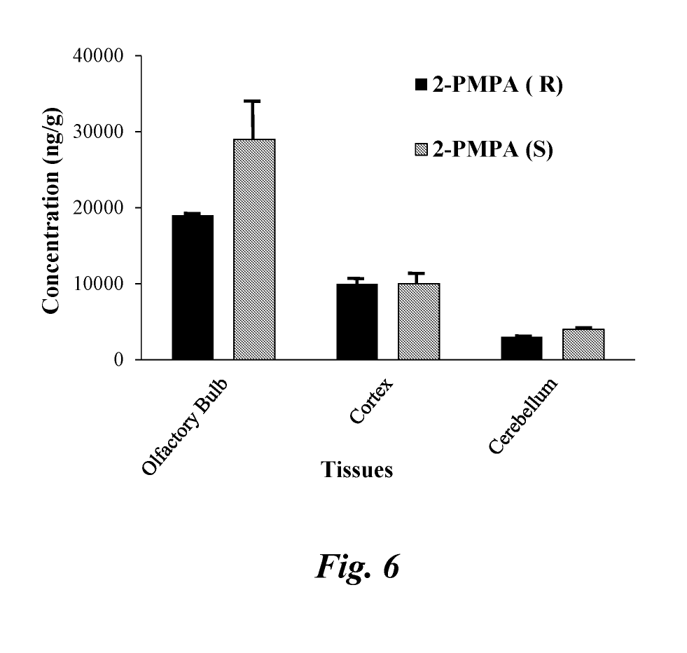

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

GCP-II is involved in the hydrolysis of the abundant neuropeptide N-acetylaspartylglutamate (NAAG) to N-acetylaspartate (NAA) and L-glutamate. Small molecule GCP-II inhibitors increase brain NAAG, which activates mGluR3, decreases glutamate, and provide therapeutic utility in a variety of preclinical models of neurodegenerative diseases wherein excess glutamate is presumed pathogenic. No inhibitor to date, however, has shown good brain penetrability.

Intranasal (i.n.) delivery of an agent to a subject, including systemic delivery through the bloodstream or, more particularly, to the brain and/or peripheral nervous system, is non-invasive and offers several advantages including avoidance of hepatic first pass clearance, rapid onset of action, frequent self-administration and easy dose adjustments (Baker and Genter, 2003). Intranasal administration of a number of small molecules, macromolecules, gene vectors and cells has been shown to be successful in animal and clinical studies (Dhuria et al., 2009; Frey et al., 1997; Chen et al., 1998; Vaka et al., 2009; Lochhead and Thorne, 2011; Johnson et al., 2010; Stevens et al., 2011). Small molecules have an added advantage of being absorbed paracellularly through the nasal epithelium after which, these molecules can then directly enter the CNS through the olfactory or the trigeminal nerve associated pathway (Baker and Genter, 2003). Small molecules like Lidocaine, Losartan, Deferoxamine, and Remoxipride have shown to be directly transported to the brain upon intranasal administration (Stevens et al., 2011; Febbraro et al., 2013; Guo et al., 2013; Hanson et al., 2009).

The presently disclosed subject matter discloses, for the first time, the intranasal route for drug delivery of GCP-II inhibitors, which results in significant enhancement of brain penetration. The presently disclosed subject matter unexpectedly discloses the identification of a 100-fold improvement in the brain-to-plasma ratio of one of the most potent, selective and widely studied GCP-II inhibitors, 2-phosphonomethyl pentanedioic acid (2-PMPA) (IC₅₀=300 pM) when administered through an intranasal route. Although the intranasal route has been employed for other small molecules, this very large (>100-fold) enhancement was unexpected as it is extremely hydrophilic (c Log p=−1.512). Also unexpectedly, other classes of GCPII inhibitors, while they also showed enhanced brain delivery, did not show as much improvement as 2-PMPA.

### I. Methods for Systemic Delivery of a GCP-II Inhibitor Via an Intranasal Route

In some embodiments, the presently disclosed subject matter provides a method for systemic delivery of a glutamate carboxypeptidase II (GCP-II) inhibitor to a subject via an intranasal route, including delivery to the brain and/or peripheral nervous system. In particular embodiments, the GCP-II inhibitor is urea, hydroxamate, thiol, or phosphonate based. In more particular embodiments, the GCP-II inhibitor is selected from the group consisting of (N—[N—[(S)-1,3-dicarboxypropyl]carbamoyl]-L-cysteine) (DCMC), 2-(3-mercaptopropyl)pentane-dioic acid (2-MPPA) and 2-(phosphonomethyl)-pentanedioic acid (2-PMPA), and stereoisomers and prodrugs thereof. In yet more particular embodiments, the GCP-II inhibitor is 2-(phosphonomethyl)-pentanedioic acid (2-PMPA), and stereoisomers and prodrugs thereof. The corresponding deuterated forms of the GCP-II inhibitors disclosed herein are also envisioned for use in the presently disclosed methods.

Prodrugs of the compounds described herein are those compounds that readily undergo chemical changes under physiological conditions to provide the compounds of the present disclosure. In particular embodiments, prodrugs of the one of more of the carboxylic acid groups of the presently disclosed compounds can be prepared. Representative promoiety groups include, but are not limited to esters, including alkyl and aryl esters, carbonates, carbamates, and amides.

Further, the presently disclosed compounds, in some embodiments, may possess asymmetric carbon atoms (optical or chiral centers). Unless otherwise stated, structures depicted herein are also meant to include all stereochemical forms of the structure, i.e., the R- and S-configurations, for each asymmetric center. Therefore, single stereochemical isomers, as well as enantiomeric and diastereomeric mixtures of the present compounds are within the scope of the disclosure. Accordingly, the present disclosure is meant to include compounds in racemic, scalemic, and optically pure forms.

In some embodiments, the presently disclosed method results in an increase in total brain concentration and an increase in brain-to-plasma partition ratio of the GCP-II inhibitor as compared to using an intraperitoneal route. In other embodiments, there is an approximately 100-fold or more increase in the brain-to-plasma partition ratio as compared to using an intraperitoneal route. In still other embodiments, most of the GCP-II inhibitor reaches the brain through the olfactory pathway, such as more than 50%, more than 60%, more than 70%, more than 80%, or more than 90%.

As used herein, the term “GCP-II” refers to a naturally occurring or endogenous GCP-II and to proteins having an amino acid sequence which is the same as that of a naturally occurring or endogenous GCP-II (e.g., recombinant proteins). Accordingly, as defined herein, the term includes mature GCP-II, glycosylated or unglycosylated GCP-II proteins, polymorphic or allelic variants, and other isoforms of GCP-II (e.g., produced by alternative splicing or other cellular processes).

As used herein, an “inhibitor” of GCP-II is a molecule that generally inhibits or decreases the activity of GCP-II. In some embodiments, small molecule GCP-II inhibitors, directly or indirectly, increase extracellular NAAG and decrease extracellular glutamate. The inhibitor may interact with GCP-II directly or may interact with another molecule that results in a decrease in the activity of GCP-II.

As used herein, the term administrating via an “intranasal route” refers to administering by way of the nasal structures. It has been found that the presently disclosed small molecule GCP-II inhibitors are much more effective at penetrating the brain when administered intranasally.

As used herein, the term “systemic delivery” includes delivery affecting the entire body, for example, delivery of an agent to the bloodstream where it can reach can affect cells throughout the body.

As used herein, the term “peripheral nervous system” includes the part of the nervous system comprising the nerves and ganglia on the outside of the brain and spinal cord. The peripheral nervous system connects the central nervous system to the limbs and organs and acts as a communication relay between the brain and the extremities. The presently disclosed small molecule GCP-II inhibitors can access the peripheral nervous system through the blood.

In some embodiments, the brain and/or peripheral nervous system, or other organ or system of interest, of the subject has excess GCP-II activity before the GCP-II inhibitor is administered. The presently disclosed subject matter shows that there is a marked elevation or excess of GCP-II activity in subjects with certain diseases or conditions. As used herein, the term “excess GCP-II activity” means an increase of GCP-II activity in a subject with a disease or condition as compared to the GCP-II activity in a subject without a similar disease or condition, such as an increase of approximately 50%, 100%, 200%, 300%, 400%, 500%, or more.

In some embodiments, performing the presently disclosed method results in inhibiting the excess GCP-II activity. In other embodiments, performing the presently disclosed method results in almost 100% inhibition of GCP-II enzyme activity in the olfactory bulb and cortex of the brain and at least 70% inhibition in the cerebellum of the brain. As used herein, the term “inhibit” means to decrease or diminish the excess GCP-II activity found in a subject. The term “inhibit” also may mean to decrease, suppress, attenuate, diminish, arrest, or stabilize the development or progression of a disease, disorder, or condition. Inhibition may occur, for e.g., by at least 10%, 20%, 30%, 40%, 50%, 60%, 70%, 80%, 90%, 95%, 98%, 99%, or even 100% compared to an untreated control subject or a subject without the disease or disorder.

As used herein, in general, the “effective amount” of an active agent refers to an amount sufficient to produce the desired effect, such as delivering the amount of active agent that can be detected in the subject, including in the brain and/or peripheral nervous system, or used for imaging, diagnosing, and/or treating the brain and/or peripheral nervous system or other organ or system of interest. A “therapeutically effective amount” of a therapeutic agent refers to the amount of the agent necessary to elicit the desired biological response. As will be appreciated by those of ordinary skill in the art, the effective amount of an agent may vary depending on such factors as the desired biological endpoint, the agent to be delivered, the composition of the pharmaceutical composition, the target tissue or cell, and the like. In some embodiments, the term “effective amount” refers to an amount sufficient to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

### II. Methods for Treating a Neurological or Psychiatric Disease or Disorder by Intranasal Administration of a GCP-II Inhibitor

In some embodiments, the presently disclosed subject matter provides a method for treating a neurological or psychiatric disease or disorder by intranasal administration of a GCP-II inhibitor.

Accordingly, in some embodiments, the presently disclosed subject matter provides a method for treating a neurological disease or disorder in a subject in need of treatment thereof, the method comprising intranasally administering to the subject a therapeutically effective amount of glutamate carboxypeptidase II (GCP-II) inhibitor, including in some embodiments, an effective amount for delivery to the brain and/or to the peripheral nervous system, e.g., through the bloodstream. In other embodiments, the GCP-II inhibitor is urea, hydroxamate, thiol, or phosphonate based. In still other embodiments, the GCP-II inhibitor is selected from the group consisting of (N—[N—[(S)-1,3-dicarboxypropyl] carbamoyl]-L-cysteine) (DCMC), 2-(3-mercaptopropyl)pentane-dioic acid (2-MPPA) and 2-(phosphonomethyl)-pentanedioic acid (2-PMPA), and stereoisomers and prodrugs thereof. In further embodiments, the GCP-II inhibitor is 2-PMPA, and stereoisomers and prodrugs thereof.

In some embodiments, the method results in an increase in total brain concentration and an increase in brain-to-plasma partition ratio of the GCP-II inhibitor as compared to using an intraperitoneal route. In other embodiments, there is an approximately 100-fold or more increase in the brain-to-plasma partition ratio using the intranasal route as compared to using an intraperitoneal route. In still other embodiments, most of the GCP-II inhibitor reaches the brain through the olfactory pathway.

In some embodiments, the neurological disease or disorder is selected from the group consisting of traumatic spinal cord and brain injury, stroke, neuropathic and inflammatory pain, neurological disorder as a result of drug abuse, epilepsy, amyotrophic lateral sclerosis (ALS), schizophrenia, Huntington's disease, neuropathy, multiple sclerosis, cognition impairment, brain cancer, HIV-associated neurocognitive disorder, and cognition impairment associated with neurodegenerative or neuropsychiatric conditions. In other embodiments, the neurological disease or disorder results in excess GCP-II activity in the brain of the subject. In still other embodiments, performing the method results in inhibiting the excess GCP-II activity. In further embodiments, performing the method results in almost 100% inhibition of GCP-II enzyme activity in the olfactory bulb and cortex of the brain and at least 70% inhibition in the cerebellum of the brain.

As used herein, the term “disease or disorder” in general refers to any condition that would benefit from treatment with a compound against one of the identified targets, or pathways, including any disease, disorder, or condition that can be treated by an effective amount of a compound against one of the identified targets, or pathways, or a pharmaceutically acceptable salt thereof.

As used herein, the terms “treat,” “treating,” “treatment,” and the like refer to reducing or ameliorating a disease or condition, and/or symptoms associated therewith. It will be appreciated that, although not precluded, treating a disease or condition does not require that the disorder, condition or symptoms associated therewith be completely eliminated. In general, the presently disclosed methods result in a decrease in the severity of a disease or condition in a subject.

For intranasal delivery, in addition to the active ingredients, pharmaceutical compositions may contain suitable pharmaceutically acceptable carriers comprising excipients and auxiliaries which facilitate processing of the active compounds into preparations which can be used pharmaceutically. The agents of the disclosure may be formulated by methods known to those of skill in the art, and may include, for example, but not limited to, examples of solubilizing, diluting, or dispersing substances, such as saline, preservatives, such as benzyl alcohol, absorption promoters, and fluorocarbons. Optimized formulations for intranasal delivery may include addition of permeability enhancers (mucoadhesives, nanoparticles, and the like) as well as combined use with an intranasal drug delivery device (for example, one that provides controlled particle dispersion with particles aerosolized to target the upper nasal cavity).

In particular, polymer-based nanoparticles, including chitosan, maltodextrin, polyethylene glycol (PEG), polylactic acid (PLA), polylactic-co-glycolic acid (PLGA), and PAMAM dendrimer; gels, including poloxamer; and lipid-based formulations, including glycerol monocaprate (Capmul™), mixtures of mono-, di-, and triglycerides and mono- and di-fatty esters of PEG (Labrafil™), palmitate, glycerol monostearate, and phospholipids can be used to administer the presently disclosed GCP-II inhibitors intranasally. See, e.g., van Woensel et al., 2013.

The presently disclosed GCP-II inhibitors also can be administered intranasally via mucoadhesive agents. Mucoadhesion is commonly defined as the adhesion between two materials, at least one of which is a mucosal surface. More particularly, mucoadhesion is the interaction between a mucin surface and a synthetic or natural polymer. Mucoadhesive dosage forms can be designed to enable prolonged retention at the site of application, providing a controlled rate of drug release for improved therapeutic outcome. Application of dosage forms to mucosal surfaces may be of benefit to drug molecules not amenable to the oral route, such as those that undergo acid degradation or extensive first-pass metabolism. Mucoadhesive materials suitable for use with nasal administration of the presently disclosed GCP-II inhibitors include, but are not limited to, soluble cellulose derivatives, such as hydroxypropyl methylcellulose (HPMC), hydroxypropyl cellulose (HPC), methylcellulose (MC), and carboxymethyl cellulose (CMC), and insoluble cellulose derivatives, such as ethylcellulose and microcrystalline cellulose (MCC), starch (e.g., Amioca®), polyacrylates, such as poly(acrylic acid) (e.g., Carbopol® 974P), functionalized mucoadhesive polymers, such as polycarbophil, hyaluronan, and amberlite resin, and chitosan (2-amino-2-deoxy-(1→4)-β-d-glucopyranan) formulations and derivatives thereof.

In some embodiments, the formulation also includes a permeability enhancer. As used herein, the term “permeability enhancer” refers to a substance that facilitates the delivery of a drug across mucosal tissue. The term encompasses chemical enhancers that, when applied to the mucosal tissue, render the tissue more permeable to the drug. Permeability enhancers include, but are not limited to, dimethyl sulfoxide (DMSO), hydrogen peroxide (H₂O₂), propylene glycol, oleic acid, cetyl alcohol, benzalkonium chloride, sodium lauryl sulphate, isopropyl myristate, Tween 80, dimethyl formamide, dimethyl acetamide, sodium lauroylsarcosinate, sorbitan monolaurate, methylsulfonylmethane, Azone, terpenes, phosphatidylcholine dependent phospholipase C, triacyl glycerol hydrolase, acid phosphatase, phospholipase A2, concentrated saline solutions (e.g., PBS and NaCl), polysorbate 80, polysorbate 20, sodium dodecanoate (C12), sodium caprate (C10) and/or sodium palmitate (C16), tert-butyl cyclohexanol (TBCH), and alpha-terpinol.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Depending upon the particular condition, or disease state, to be treated or prevented, additional therapeutic agents, which are normally administered to treat or prevent that condition, may be administered together with the inhibitors of this disclosure. These additional agents may be administered separately, as part of a multiple dosage regimen, from the inhibitor-containing composition. Alternatively, these agents may be part of a single dosage form, mixed together with the inhibitor in a single composition.

The compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

### III. Methods for Diagnosing a Neurological or Psychiatric Disease or Disorder by Intranasal Administration of a GCP-II Inhibitor

In some embodiments, the presently disclosed methods provide for diagnosing a neurological or psychiatric disease or disorder by administering through an intranasal route a high affinity binder, such as a GCP-II inhibitor, labeled with a fluorescent, luminescent, phosphorescent, radioactive, or colorimetric compound for imaging purposes.

Accordingly, in some embodiments, the presently disclosed subject matter provides a method for diagnosing a neurological disease or disorder involving alteration of glutamate carboxypeptidase II enzyme (GCP-II) levels or location in the brain and/or peripheral nervous system of a subject, the method comprising intranasally administering to the subject an effective amount of GCP-II inhibitor labeled with a fluorescent species or radiolabeled with an isotope and obtaining an image of the brain and/or peripheral nervous system of the subject, wherein an alteration in levels or location of GCP-II in the brain and/or peripheral nervous system as compared to the brain and/or peripheral nervous system of a subject without the neurological disease or disorder is indicative that the subject has the neurological disease or disorder.

As used herein, the term “diagnosis” refers to a predictive process in which the presence, absence, severity or course of treatment of a disease, disorder or other medical condition is assessed. For purposes herein, diagnosis also includes predictive processes for determining the outcome resulting from a treatment. Likewise, the term “diagnosing,” refers to the determination of whether a sample specimen exhibits one or more characteristics of a condition or disease. The term “diagnosing” includes establishing the presence or absence of, for example, a target molecule, such as GCP-II, or reagent bound target molecule, or establishing, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease. The term “prognosis” and derivations thereof, refers to the determination or prediction of the course of a disease or condition. The course of a disease or condition can be determined, for example, based on life expectancy or quality of life. “Prognosis” includes the determination of the time course of a disease or condition, with or without a treatment or treatments. In the instance where treatment(s) are contemplated, the prognosis includes determining the efficacy of a treatment for a disease or condition. The term “monitoring,” such as in “monitoring the course of a disease or condition,” refers to the ongoing diagnosis of samples obtained from a subject having or suspected of having a disease or condition.

In some embodiments, the GCP-II inhibitor is urea, hydroxamate, thiol, or phosphonate based. In other embodiments, the GCP-II inhibitor is selected from the group consisting of (N—[N—[(S)-1,3-dicarboxypropyl] carbamoyl]-L-cysteine) (DCMC), 2-(3-mercaptopropyl)pentane-dioic acid (2-MPPA) and 2-(phosphonomethyl)-pentanedioic acid (2-PMPA), and stereoisomers and prodrugs thereof. In still further embodiments, the GCP-II inhibitor is 2-PMPA, and stereoisomers and prodrugs thereof.

In some embodiments, the method results in an increase in total brain concentration and an increase in brain-to-plasma partition ratio of the GCP-II inhibitor as compared to using an intraperitoneal route. In other embodiments, there is an approximately 100-fold or more increase in the brain-to-plasma partition ratio using the intranasal route as compared to using an intraperitoneal route. In still other embodiments, most of the GCP-II inhibitor reaches the brain through the olfactory pathway.

In some embodiments, the presently disclosed methods can be used to diagnose a neurological disease or disorder. In other embodiments, the neurological disease or disorder is selected from the group consisting of traumatic spinal cord and brain injury, stroke, neuropathic and inflammatory pain, neurological disorder as a result of drug abuse, epilepsy, amyotrophic lateral sclerosis (ALS), schizophrenia, Huntington's disease, neuropathy, multiple sclerosis, cognition impairment, brain cancer, HIV-associated neurocognitive disorder, and cognition impairment associated with neurodegenerative or neuropsychiatric conditions. In still other embodiments, the neurological disease or disorder results in excess GCP-II activity in the brain of the subject.

In some embodiments, the GCP-II inhibitor is labeled with a fluorescent compound. The fluorescent compound may be selected from the available compounds which have known fluorescent characteristics, i.e. which emit fluorescent light at an emission wavelength when illuminated with light of a different, shorter, excitation wavelength. In other embodiments, the GCP-II inhibitor is radiolabeled with an isotope, wherein the isotope is selected from the group consisting of ¹²⁵I, ¹²³I, ¹⁸F ¹⁴C, and ⁶⁸Ga.

### IV. Methods for Imaging GCP-II by Intranasal Administration of a GCP-II Inhibitor

In some embodiments, the presently disclosed subject matter may be used to image GCP-II in the brain and/or peripheral nervous system, or any other organ or system or interest, of a subject when the subject is not being diagnosed for a neurological or psychiatric disorder. Accordingly, in some embodiments, the presently disclosed subject matter provides a method for imaging glutamate carboxypeptidase II (GCP-II) in a subject, the method comprising intranasally administering to the subject an effective amount of GCP-II inhibitor labeled with a fluorescent species or radiolabeled with an isotope and obtaining an image of the subject, including an image of, in some embodiment, the brain and/or peripheral nervous system. In other embodiments, the GCP-II inhibitor is urea, hydroxamate, thiol, or phosphonate based. In still other embodiments, the GCP-II inhibitor is selected from the group consisting of (N—[N—[(S)-1,3-dicarboxypropyl] carbamoyl]-L-cysteine) (DCMC), 2-(3-mercaptopropyl)pentane-dioic acid (2-MPPA) and 2-(phosphonomethyl)-pentanedioic acid (2-PMPA), and stereoisomers and prodrugs thereof. In further embodiments, the GCP-II inhibitor is 2-(phosphonomethyl)-pentanedioic acid (2-PMPA), and stereoisomers and prodrugs thereof. In still further embodiments, the isotope is selected from the group consisting of ¹²⁵I, ¹²³I, ¹⁸F, ¹⁴C, and ⁶⁸Ga.

### V. General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Materials and Methods

Synthesis of Inhibitors:

2-PMPA, 2-PMSA (internal standard), and 2-MPPA and were synthesized internally as reported previously (Jackson et al., 2001; Vitharana et al., 2002). DCMC was donated by Dr. Martin Pomper at The Johns Hopkins University. Losartan (internal standard) was obtained from Sigma-Aldrich (St. Louis, Mo.). LC/MS grade acetonitrile and water (LC/MS grade) with 0.1% formic acid were obtained from Fisher Scientific (Hanover Park, Ill.). Drug-free (blank) heparinized rat plasma was obtained from Innovative Research Inc. (Plymouth, Minn.). All other chemical and reagents were purchased from Sigma-Aldrich (St. Louis, Mo.).

Animal Studies:

All of the animal studies in rodents were performed as per protocols approved by the Institutional Animal Care and Use Committee (Protocol# RA13) at The Johns Hopkins University and primate studies were conducted in accordance with the guidelines recommended in Guide for the Care and Use of Laboratory Animals (National Academy Press, Washington D.C., 2011) following approval by Animal Care and Use Committee (Protocol #031637) at Ricerca Biosciences (Concord, Ohio, USA).

Rodent i.n. and i.p. Dosing:

Studies were conducted in male Wistar rats (6-8 weeks; weighing between 200 gm to 250 gm) obtained from Harlan® Laboratories (Indianapolis, Ind.) that were maintained in a controlled environment and allowed food and water ad libitum. Intranasal administrations were performed according to previously described methods with minor modifications. Briefly, rats were anesthetized with a 1-1.5 mL intraperitoneal (i.p.) dose of 10% chloral hydrate (approved under the protocol RA#13), and kept under anesthesia with additional chloral hydrate as needed throughout the entire experiment. To prevent drainage of nasally dosed solution, the nasal cavity was isolated from the respiratory and gastrointestinal tracts. An incision was made along the neck, and the trachea isolated and transected. The upper part was tied off with a 3-0 silk suture, and the lower part cannulated with PE240 tubing to aid air breathing. Rats were maintained lying on their back, and in this position, given 10 μL (375 mg/mL) of the experimental drug solution per nostril using a micro-syringe connected to 1.5 cm PE-10 tube. The total dose received was 30 mg/kg for each drug solution.

For i.p. studies, 2-PMPA was administered as a single i.p. dose. All dosing solutions were prepared on the day of the experiment in 50 mM HEPES buffered saline, and pH adjusted to 7.4 before injection. At various time points following drug administration (0.16 h, 0.5 h, 1 h, 3 h, 5 h) post dose, animals (n=3 per time point, except 3 h n=2 animals) were euthanized with CO₂, and blood samples were collected in heparinized microtubes by cardiac puncture and tissues (olfactory bulb, frontal cortex, and cerebellum) were dissected and immediately flash frozen (−80° C.). Plasma was prepared by centrifugation immediately after collection of blood samples. All samples were stored in −80° C. until bioanalysis.

Rodent Ex Vivo GCP-II Enzymatic Activity:

One half of the brain tissues collected following i.n. administration (1 h post dose) was used to determine GCP-II NAAG hydrolyzing activity. In brief, tissues were weighed and immersed in 0.5 mL of ice-cold 50 mM Tris Buffer (pH 7.7 at RT). Each tissue was sonicated for 30-60 seconds using an ultrasonic cell disrupter. After a 2 minute spin at 13,000 g, supernatants were analyzed for protein content and NAAG-hydrolyzing activity measurements were performed as previously described (Rojas et al., 2012; Robinson et al., 1987).

Non-Human i.n. Primate Dosing:

The study was conducted in accordance with the guidelines recommended in Guide for the Care and Use of Laboratory Animals (National Academy Press, Washington D.C., 2011). A male cynomolgus monkey (approximately 3.5 kg, non-drug naive) was housed in a stainless steel cage (size 30″ (76.2 cm) wide×31″ (78.74 cm) deep×31.5″ (80.01 cm) high) maintaining temperature of 64-84° F. (17.8° C.-28.9° C.), humidity of 30-70% with alternating 12-hour light/dark cycle as per the USDA Animal Welfare Act (9 CFR, Parts 1, 2, and 3). Food was provided twice daily in amounts appropriate for the size and age of the animals and tap water was available ad libitum. To provide psychological enrichment, the monkey was provided television entertainment for at least 1 hour per day, (at least 2 to 3 times weekly); received fruits, vegetables, and additional treats minimally 3 times weekly; and housed with rubber toys on a full-time basis throughout the duration of the study. The health status of the animal was evaluated in accordance with accepted veterinary practice; no abnormalities were observed throughout the study. Following the last sample collection, the animal was released to the facility stock animal colony. The animal was healthy and was not sacrificed. The study was conducted by Michael Stonerook, Ph.D., D.V.M., DABT, the technical director at Ricera Biosciences, LLC.

The monkey was sedated with 45 mg of ketamine and 0.25 mg of midazolam given as an intramuscular injection prior to test article administration. Sedation was maintained through blood and cerebrospinal fluid (CSF) sample collections with ketamine/midazolam at a starting rate of 20 mg/kg/hr ketamine and 0.4 mg/kg/hr midazolam. 2-PMPA was administered as an aqueous solution (similar to rodent studies) via i.n. delivery employing a drug delivery device (Kurve Technology, Bothell, Wash.), designed to deliver drugs to the olfactory region to maximize transport to the central nervous system. The device was actuated for a period of 2 min in one nostril (depositing 100 μL). The nose piece was cleaned with a mist of air and then the same procedure was performed in the second nostril (100 μL). Total dose delivered was 100 mg. CSF sample (target of 50 μL) was obtained by an indwelling cannula placed in the intrathecal space at the cisterna magna at 0.5 h post dose. Blood was collected via venipuncture of the femoral vein at 0.5 h post dose and plasma was obtained by low speed centrifugation at 1500×g for 10 minutes. The plasma was flash-frozen on dry ice after separation. Plasma and CSF samples were stored in a freezer set at −70° C., until bioanalysis.

Bioanalysis of DCMC, 2-MPPA, and 2-PMPA in Rodent Plasma and Brain:

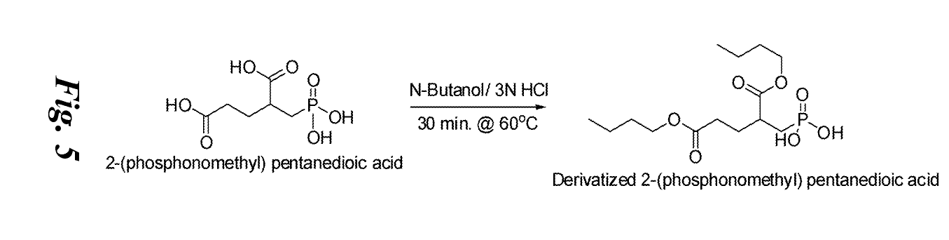

For quantification of analytes in plasma and brain tissues, the extractions were performed using protein precipitation and subsequently processed for analysis by LC/MS/MS. Briefly, prior to extraction, frozen samples were thawed on ice. For plasma samples, 50 μL of the calibration standard or sample were transferred into silanized vials. For brain tissues, the samples were weighed in a 1.7 mL silanized vials to which 4 times the volume of methanol (dilution 1:5) was added. The tissues were stored in −20° C. for 1 h and then homogenized. The calibration curves were developed using plasma and brain from untreated animals as a matrix. For plasma, sample preparation involved a single liquid extraction by addition of 300 μL of methanol as extraction solution with internal standard, followed by vortexing for 30 s and then centrifugation at 12000×g for 10 min. Supernatant was transferred and evaporated to dryness at 40° C. under a gentle stream of nitrogen. For brain tissue, homogenized samples were vortexed and centrifuged as above, and 100 μL supernatant was mixed with 100 μL of internal standard in methanol, and then evaporated to dryness at 40° C. under a gentle stream of nitrogen. For 2-PMPA analysis, samples were derivatized to improve sensitivity and enable reverse phase chromatography as shown in FIG. 5. For derivatization, the residue was reconstituted with 100 μL of n-butanol with 3N HCl and samples were vortexed. The samples were heated at 60° C. in a shaking water bath for 30 min. At the end of 30 min, the derivatized samples were dried under a gentle stream of nitrogen. 2-MPPA and DCMC were processed without additional derivatization step and were amenable to reverse phase chromatography. Following extraction of 2-MPPA, DCMC, and derivatized 2-PMPA, the residue was reconstituted in 100 μL of 30% acetonitrile and water v/v. The samples were vortexed and centrifuged. Supernatant (75 μL) was transferred to a 250 μL polypropylene autosampler vial sealed with a Teflon cap and a volume of 10 μL was injected onto the ultra11 performance liquid chromatography (UPLC) instrument for quantitative analysis using a temperature-controlled autosampler operating at 10° C.

Chromatographic analysis was performed using an Accela™ ultra high-performance system consisting of an analytical pump, and an autosampler coupled with TSQ Vantage mass spectrometer (Thermo Fisher Scientific Inc., Waltham Mass.). Separation of the analyte was achieved at ambient temperature using Agilent Eclipse Plus UPLC column (100×2.1 mm i.d.) packed with a 1.8 μm C18 stationary phase. The mobile phase was composed of 0.1% formic acid in acetonitrile and 0.1% formic acid in H₂O with gradient elution. The total run time for each analyte was 5.0 min. The [M⁺H]⁺ ion transitions of derivatized 2-PMPA (m/z 339.537>191.354, 149.308), and the internal standard (m/z 25.522>121.296,195.345); DCMC (m/z 309.416>119.272,130.321) and the internal standard (335.460>145.28, 188.32) and the [M-H]-ion transitions for 2-MPPA, at (m/z 205.300>171.459, 187.462) and the internal standard (m/z 421.670>127.368, 179.576) were monitored by LC/MS/MS. Calibration curves over the range of 0.034-17.0 μg/mL for DCMC; and 0.021-10.3 μg/mL for 2-MPPA in brain tissue; and 0.011-22.6 μg/mL for 2-PMPA in plasma and tissue were constructed from the peak area ratio of the analyte to the internal standard using linear regression with a weighting factor of 1/(nominal concentration). Correlation coefficient of greater than 0.99 was obtained in all analytical runs for all analytes. The mean predicted relative standard deviation for back calculated concentrations of the standards for all analytes were within the range of 85 to 115%, except for the lowest concentration which was within the range of 80 to 120%.

Pharmacokinetic Analysis of 2-PMPA in Rodents:

Mean plasma and tissue concentrations of 2-PMPA were analyzed using non-compartmental method as implemented in the computer software program WinNonlin Professional version 5.0.1 (Pharsight Corp., Mountain View, Calif.). The maximum plasma concentration (Cₘₐₓ) and time to Cₘₐₓ (Tₘₐₓ) were the observed values. The area under the plasma concentration time curve (AUC) value was calculated to the last quantifiable sample (AUCₗₐₛₜ) by use of the log linear trapezoidal rule. The brain-to-plasma partition coefficients were calculated as a ratio of their AUCs (AUC_{0-t, brain}/AUC_{0-t, plasma}). The elimination half-life (t_{1/2}) was determined by dividing 0.693 by λ_{z}.

Analysis of 2-PMPA in Primate Plasma and CSF:

For analysis of 2-PMPA in CSF samples, no derivatization was performed as CSF offers a much cleaner matrix compared to brain and plasma which are complex. Standard curves were generated in blank artificial CSF. Calibration curves were constructed in the range of 0.113-22.6 μg/mL. Samples were analyzed on Agilent QTOF mass spectrometer by LC/MS. Samples (20 μL) were injected and separated on an Agilent 1290 LC equipped with an Agilent Eclipse Plus C18 column (2.1×100 mm) packed with 1.8 micron stationary phase. The mobile phase consisted of 0.1% formic acid in water and 0.1% formic acid in acetonitrile with an isocratic elution at 2.5% organic. Analytes were detected with an Agilent 6520 QTOF mass spectrometer in negative mode using [M-H]− ions for 2-PMPA (225.0163) and the internal standard (325.1043). Calibration curves were generated with a correlation coefficient >0.99 in a similar manner as described above. Plasma analysis was conducted in a similar manner as described above (rodent plasma 2-PMPA analysis) using naïve male cynomolgus monkey plasma for standard curve.

### Example 2

### Brain Concentrations of I.N. Administered DCMC, 2-MPPA and 2-PMPA in Rodents

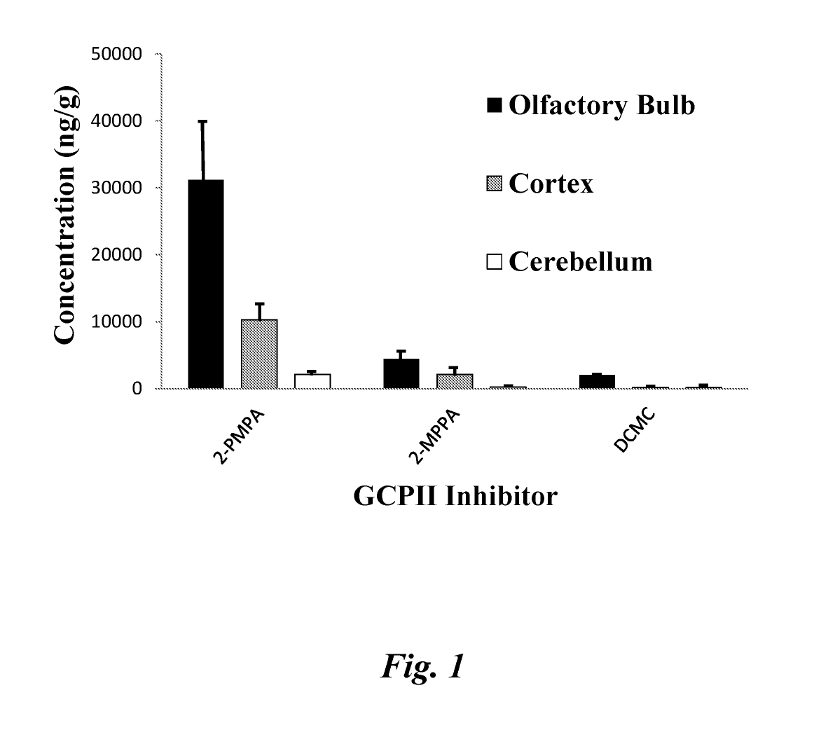

The structures of three chemically distinct GCP-II inhibitors DCMC, 2-MPPA and 2-PMPA and their IC₅₀ values are shown in Table 1. DCMC, 2-MPPA and 2-PMPA were evaluated in a single time point (1 hr post dose) pharmacokinetic study in rats dosed i.n. at 30 mg/kg. While all three inhibitors showed some brain penetration, 2-PMPA exhibited the highest levels (FIG. 1). As shown in FIG. 1, at 1 hr following i.n. administration, 2-PMPA was found in the olfactory bulb, cortex and cerebellum at 31.2 μg/g, 10.3 μg/g and 2.13 μg/g respectively. 2-MPPA and DCMC showed less exposure with 4.46 μg/g and 2.12 μg/g, 0.26 μg/g and 2.03 μg/g, and 0.21 μg/g and 0.20 μg/g in the olfactory bulb, cortex and cerebellum, respectively.

TABLE 1
Chemical structures and IC₅₀ values of DCMC, 2-MPPA, 2-PMPA

| | Structural | | Potency |
|---|---|---|---|
| Name | Class | Structure | (IC₅₀) |
| DCMC (N-[N-[(S)-1,3- dicarboxypropyl] carbamoyl]-L- cysteine) | Urea | 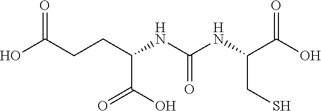 [SMILES: O=C(O)CC[C@H](NC(=O)N[C@@H](CS)C(=O)O)C(=O)O] | 3.1 nM |
| 2-MPPA 2-(3-mercaptopropyl) pentane-dioic acid | Thiol | 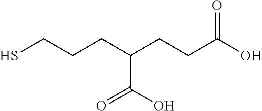 [SMILES: O=C(O)CCC(CCCS)C(=O)O] | 90 nM |
| 2-PMPA 2-(phosphonomethyl)- pentanedioic acid | Phosphonate | 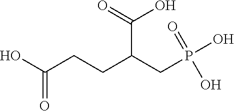 [SMILES: O=C(O)CCC(CP(=O)(O)O)C(=O)O] | 0.3 nM |

### Example 3

### Comparison of the Pharmacokinetics of I.P. Versus I.N. Administered 2-PMPA in Rodents

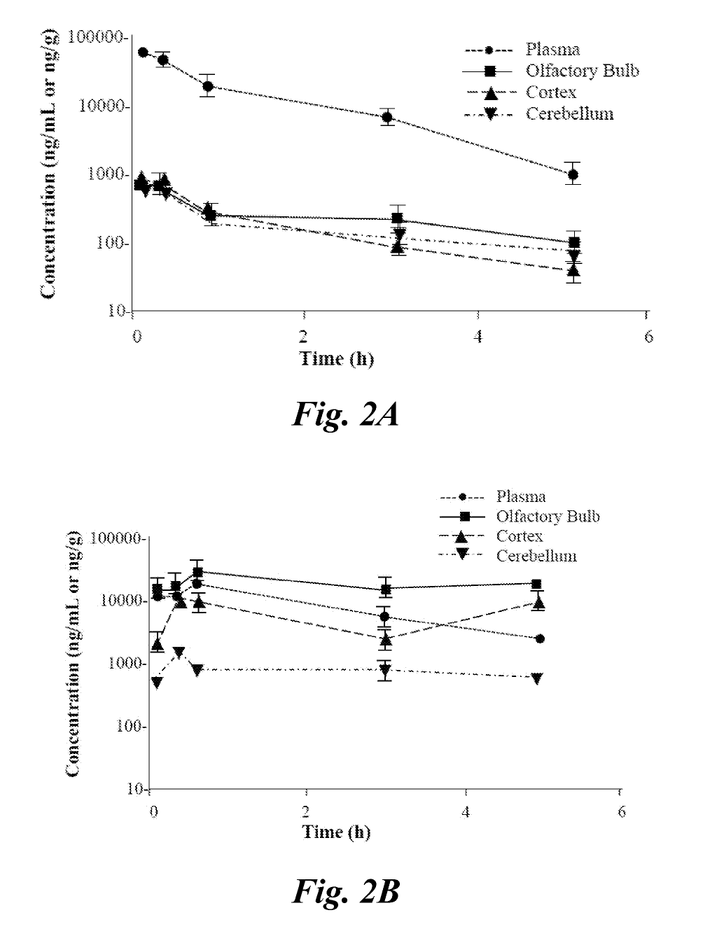

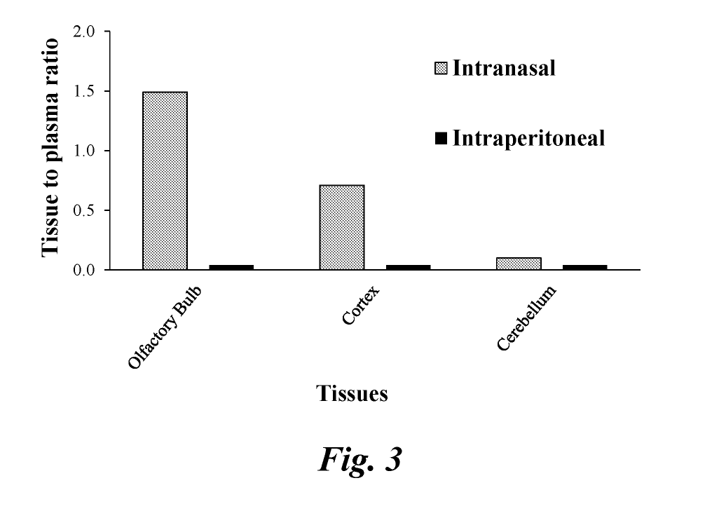

Pharmacokinetic studies of 30 mg/kg 2-PMPA in rat plasma and brain tissues following i.n. and i.p. administration were conducted and compared. Similar to what has been previously demonstrated (Rais et al., 2014), i.p. administered 2-PMPA showed rapid absorption in plasma with peak plasma concentration (Cₘₐₓ) of 49.5 μg/mL observed at the first time point of 0.167 h. The AUC₀₋ₜ achieved for plasma was 50.3 h*μg/mL and the elimination t½ value was 0.99 h depicting rapid elimination. The apparent volume of distribution was low (0.82 L/kg) and the apparent clearance was rapid (9.71 mL/min/kg). As shown in FIG. 2A and FIG. 2B, the AUC₀₋ₜ achieved for olfactory bulb, cortex and cerebellum were 1.15 h*μg/g, 0.84 h*μg/g, and 0.80 h*μg/g respectively. The brain tissue to plasma ratios based on AUCs (AUC₀₋ₜ, brain/AUC_{0-t, plasma}) was less than 0.02 for olfactory bulb, cortex, and plasma, cerebellum (FIG. 3).

Following i.n. administration, the 2-PMPA plasma Cₘₐₓ was 24.7 μg/mL observed at 1 h post dose. The plasma AUC₀₋ₜ was 52.3 h*μg/mL. The AUC₀₋ₜ for olfactory bulb, cortex and cerebellum were 78.1 h*μg/g, 37.7 h*μg/g and 5.27 h*μg/g respectively (FIG. 2B). The brain tissue to plasma ratios based on AUCs (AUC₀₋ₜ, brain/AUC_{0-t, plasma}) were 1.49, 0.71 and 0.10 in the olfactory bulb, cortex, and cerebellum respectively (FIG. 3). The elimination t_{1/2} value and apparent clearance were not reported due to the lack of elimination phase following intranasal route.

### Example 4

### GCP-II Functional Activity in Rodent Brain Following 2-PMPA I.N. Administration

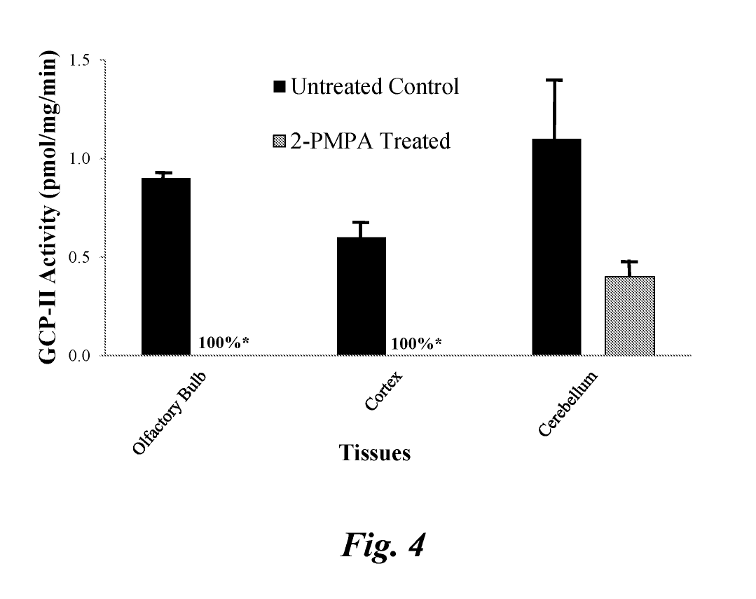

As shown in FIG. 4, target engagement studies were performed by measurement of GCP-II enzymatic activity in brain tissue 1 h following i.n. 2-PMPA administration. There was complete (100%) inhibition of GCP-II activity measured in olfactory bulb and cortex following i.n. administration and almost complete (70%±5%) inhibition in the cerebellum.

### Example 5

### CSF Exposure of 2-PMPA Following I.N. Administration in Nonhuman Primates

In an exploratory study conducted by Ricerca Biosciences, LLC, 2-PMPA was administered to a male cynomolgus monkey using the Vianase™ intranasal device at a total dose of 100 mg. Following i.n. administration at 30 min post dose, the plasma level of 2-PMPA was below the 15 limit of quantitation (<50 nM), while the CSF concentration was 0.32 μg/mL (approximately 1.504) determined by LC/MS/MS.

### Example 6

### Brain Exposure of I.N. Administered R-Versus S-2-PMPA Enantiomers

Referring now to FIG. 7, the brain penetration of purified R- and S-enantiomers of 2-PMPA were compared. In the olfactory bulb, the concentration of the S-enantiomer was higher than the R-enantiomer with 28.5 μg/g versus 19.3 μg/g respectively, but no differences were observed in the exposure of the two enantiomers in the cortex or the cerebellum. Intranasal 2-PMPA provided 10.1 μg/g and 10.4 μg/g in cortex, and 1.78 μg/g and 2.51 μg/g in the cerebellum for R- and S-2-PMPA, respectively. Given this observation, all subsequent studies were conducted with racemic 2-PMPA.

### Example 7

### Summary and Discussion

In summary, three structurally distinct classes of GCP-II inhibitors were evaluated including DCMC (urea-based), 2-MPPA (thiol-based) and 2-PMPA (phosphonate-based) as disclosed in Table 1. While all showed some brain penetration following i.n. administration, 2-PMPA exhibited the highest levels and was chosen for further evaluation. Compared to intraperitoneal (i.p.) administration, equivalent doses of i.n. administered 2-PMPA resulted in similar plasma exposures (AUC₀₋ₜ, i.n./AUC₀₋ₜ, i.p.=1.0) but dramatically enhanced brain exposures in the olfactory bulb (AUC₀₋ₜ, i.n./AUC₀₋ₜ, i.p.=67), cortex (AUC₀₋ₜ, i.n./AUC₀₋ₜ, i.p.=46) and cerebellum (AUC₀₋ₜ, i.n./AUC₀₋ₜ, i.p.=6.3). Following i.n. administration, the brain tissue to plasma ratio based on AUC₀₋ₜ in the olfactory bulb, cortex, and cerebellum were 1.49, 0.71 and 0.10 respectively compared to an i.p. brain tissue to plasma ratio of less than 0.02 in all areas. Furthermore, i.n. administration of 2-PMPA resulted in complete inhibition of brain GCP-II enzymatic activity ex-vivo confirming target engagement. Lastly, because the rodent nasal system is not similar to humans, i.n. 2-PMPA was also evaluated in a non-human primate. It has been reported that i.n. 2-PMPA provides selective brain delivery with micromolar concentrations. These three studies support intranasal delivery of 2-PMPA to deliver therapeutic concentrations in the brain.

GCP-II (also termed NAALADase or NAAG peptidase) is a 94 kD class II membrane bound zinc metalloenzyme that modulates glutamatergic transmission through its NAAG hydrolyzing activity in the CNS. Inhibition of GCP-II has shown to provide neuroprotection both by increasing brain NAAG and modulating mGluR3 receptor activity, and by decreasing glutamate release. Potent small-molecule GCP-II inhibitors have demonstrated therapeutic utility in over twenty preclinical models of neurological disorders demonstrated independently by several laboratories.

Unfortunately, the rational design of GCP-II inhibitors with glutarate and zinc chelating moieties has resulted in poor physicochemical properties, including extreme hydrophilic nature, with limited oral bioavailability and blood-brain barrier (BBB) penetration. Rigorous efforts led to the design of thiol based inhibitors which were found to be orally bioavailable in preclinical species, of which 2-MPPA was evaluated in clinical studies. Unfortunately, its development was halted due to membranoproliferative glomerular nephritis, thought to be immune complex mediated, observed in non-human primates. As a class, thiol drugs have a known risk of inducing immunotoxicity and hypersensitivity reactions. Thus, in spite of finding numerous potent and efficacious molecules, to date no GCP-II inhibitor has advanced into clinical studies.

To overcome these challenges and to aide in the transition of potent, selective, and efficacious small molecule GCP-II inhibitors into the clinical setting, alternative patient compliant routes for CNS delivery have been examined. One such alternative non-invasive mechanism is the nasal route for delivery of drugs to the brain via the olfactory region, since the olfactory receptor cells are in direct contact with both the nasal environment and the central nervous system (CNS). As a result, delivery of biologics, peptides, and small molecules from the nasal passages to the brain have now been documented in numerous animal and clinical studies.

Intranasal drug delivery of three structurally distinct classes of GCP-II inhibitors were first assessed, including a urea, thiol and phosphonate based inhibitor namely DCMC, 2-MPPA and 2-PMPA, respectively. All three drugs shared common glutarate functionality but with a different zinc chelating group (Table 1). Of the three compounds delivered intranasally, 2-PMPA showed the highest penetration in the brain tissues followed by 2-MPPA and DCMC. 2-PMPA's preferential brain uptake was not entirely surprising since, of the inhibitors studied via the systemic route, 2-PMPA has shown low but enhanced brain penetration compared to 2-MPPA and DCMC.

Using racemic 2-PMPA, a time course evaluation following intranasal administration and directly compared it to a systemic i.p. route was then conducted. 2-PMPA has been evaluated in several preclinical models using i.p. route of administration and has generally shown efficacy at 50-100 mg/kg despite its picomolar potency in vitro. This could be explained in part due to low brain-to-plasma ratio of 2-PMPA of ≤2% following systemic administration. The results illustrate significant differences in the pharmacokinetics of 2-PMPA following i.n. versus i.p. administration. As seen in FIG. 2A and FIG. 2B, both plasma and brain tissues had detectable concentration within the first 10 min. Following the i.p. route, there was almost two orders of magnitude difference in the concentrations measured in plasma vs brain tissue suggesting low extent of partitioning into brain via systemic route similar to previous findings. When directly comparing the i.p. vs i.n. route, based on AUC₀₋ₜ, the plasma exposures from the two routes were same. A dramatic difference, however, was observed in brain penetration. Most importantly, the nasal route led not only to an increase in absolute exposures (increased total brain concentration compared to i.p. route) but also in relative exposures (increased brain-to-plasma partition ratio compared to i.p. route). Further, by pooling the data from plasma and brain following i.p. and i.n. route, it is apparent that most of the 2-PMPA reaching the brain was from direct i.n. route through the olfactory pathway and only 2% of it is accounted from the plasma 2-PMPA. Another notable difference following i.n. administration was the slower elimination compared to the i.p. route perhaps due to the existence of a slow continuing absorption process from the nasal mucosa. This profile indicates absorption rate-limited elimination (flip-flop kinetics) as has been previously described via i.n. route (Stevens et al., 2011).

These results are promising and provide a clinical path forward for an extremely hydrophilic and a potent compound like 2-PMPA, that has been highly efficacious in many preclinical models of neurological disorders, but whose translation into clinic has been hampered due to poor physiochemical properties. Most studies investigating the pathway from the nose to the brain have been performed in rodents. In comparison to the human nose, however, the rodent nose offers a significantly higher surface area to volume ratio and a significantly higher percentage of nasal epithelium devoted to olfaction. Furthermore, in humans, the olfactory region is located in the roof of the nasal cavity while the olfactory area in rats is spread throughout the posterior part of the cavity. These anatomical differences are important and should be taken into consideration for correct interpretation of results from rodent models. One model species that has been recently used and is anatomically similar to humans is non-human primates. A pilot primate study was performed using a device specifically designed for targeting the olfactory region and is also being currently employed in clinical studies. This device, known as Vianase™ developed by Kurve Inc., is the liquid drug delivery system based on Controlled Particle Dispersion technology. The exploratory study in a non-human primate using the olfactory targeting device revealed selective permeation with 1.5 μM concentrations in primate CSF and undetectable levels in plasma (<10 nM) at 30 min post dose. These data are promising representing selective delivery to the brain and >100 fold CSF concentration versus the IC₅₀ for 2-PMPA. Overall the i.n. studies suggest a direct pathway for the transfer of 2-PMPA via the olfactory mucosa into the CNS. Intranasal drug-delivery provides a strategy to achieve therapeutic 2-PMPA concentrations in the brain.

In addition to its therapeutic potential, intranasal administration of 2-PMPA also has diagnostic uses as an imaging agent in the brain, when attached to a suitable isotope for GCPII localization, including, but not limited to, ¹²⁵I, ¹²³I, ¹⁸F, ¹⁴C, and ⁶⁸Ga, or characterization of its changes in neurological disease.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Adedoyin, M. O., et al. Endogenous N-acetylaspartylglutamate (NAAG) inhibits synaptic plasticity/transmission in the amygdala in a mouse inflammatory pain model. Mol. Pain. 2010, 6: 60.
Bacich, D. J., et al. Mice lacking glutamate carboxypeptidase II are protected from peripheral neuropathy and ischemic brain injury. Journal of neurochemistry. 2005, 95: 314-323.
Baker, H.; Genter, M. B. The Olfactory System and the Nasal Mucosa as Portals of Entry of Viruses, Drugs, and Other Exogenous Agents into the Brain. In: Doty R L, editor. Handbook of Olfaction and Gustation. 2003, Boca Raton: CRC Press.
Barinka, C., et al. Glutamate carboxypeptidase II in diagnosis and treatment of neurologic disorders and prostate cancer. Curr. Med. Chem. 2012, 19: 856-870.
Berent-Spillson A., et al. Protection against glucose-induced neuronal death by NAAG and GCP II inhibition is regulated by mGluR3, Journal of neurochemistry. 2004, 89: 90-99.
Carozzi, V. A., et al. Glutamate carboxypeptidase inhibition reduces the severity of chemotherapy-induced peripheral neurotoxicity in rat. Neurotoxicity research. 2010, 7: 380-391.
Carpenter, K. J., et al. Effects of GCP-II inhibition on responses of dorsal horn neurones after inflammation and neuropathy: an electrophysiological study in the rat, Neuropeptides. 2003, 37: 298-306.
Carpenter, K. J., and Dickenson, A. H. Amino acids are still as exciting as ever. Curr. Opin. Pharmacol. 2001, 1: 57-61.
Chen, X. Q., et al. (1998) Delivery of Nerve Growth Factor to the Brain via the Olfactory Pathway. J Alzheimers Dis 1: 35-44.
Chen, S. R., et al. Effect of 2-(phosphono-methyl)-pentanedioic acid on allodynia and afferent ectopic discharges in a rat model of neuropathic pain. J. Pharmacol. Exp. Ther. 2002, 300: 662-667.
Chopra, M., et al. The neuroactive peptide Nacetylaspartylglutamate is not an agonist at the metabotropic glutamate receptor subtype 3 of metabotropic glutamate receptor. The Journal of pharmacology and experimental therapeutics. 2009, 330: 212-219.
Craft, S., et al. Intranasal insulin therapy for Alzheimer disease and amnestic mild cognitive impairment: a pilot clinical trial. Archives of neurology. 2012, 69: 29-38.
Dhuria, S. V., et al. Intranasal delivery to the central nervous system: mechanisms and experimental considerations. J. Pharm. Sci. 2009, 99: 1654-1673.
Farrell, J., et al. Characterization of sulfamethoxazole and sulfamethoxazole metabolite-specific T-cell responses in animals and humans. J. Pharmacol. Exp. Ther. 2003, 306: 229-237.
Febbraro, F., et al. Chronic intranasal deferoxamine ameliorates motor defects and pathology in the alpha-synuclein rAAV Parkinson's model. Exp. Neurol. 2013, 247: 45-58.
Foss, C. A., et al. Radiolabeled small-molecule ligands for prostate-specific membrane antigen: in vivo imaging in experimental models of prostate cancer. Clinical cancer research: an official journal of the American Association for Cancer Research. 2005, 11: 4022-4028.
Frey, W., et al. Delivery of 125I-NGF to the Brain via the Olfactory Route. Drug Deliv. 1997, 4: 87-92.
Ghadge, G. D., et al. Glutamate carboxypeptidase II inhibition protects motor neurons from death in familial amyotrophic lateral sclerosis models. Proc. Natl. Acad. Sci. U.S.A. 2003, 100: 9554-9559.
Gibaldi, M and Perrier, D. Noncompartmental Analysis Based on Statistical Moment Theory. Pharmacokinetics 1982, (ed 2) New York, N.Y., Marcel Dekker: 409-417.
Guo, C., et al. Intranasal deferoxamine reverses iron-induced memory deficits and inhibits amyloidogenic APP processing in a transgenic mouse model of Alzheimer's disease. Neurobiol. Aging. 2013, 34: 562-575.
Hanson, L. R., et al. Intranasal deferoxamine provides increased brain exposure and significant protection in rat ischemic stroke. J. Pharmacol Exp. Ther. 2009, 330: 679-686.
Hussain, A., et al. Nasal absorption of propranolol from different dosage forms by rats and dogs. J. Pharm. Sci. 1980, 69: 1411-1413.
Jackson, P. F., et al. Design and pharmacological activity of phosphinic acid based NAALADase inhibitors. J. Med. Chem. 2001, 44: 4170-4175.
Janczura, K. J., et al. NAAG peptidase inhibitors and deletion of NAAG peptidase gene enhance memory in novel object recognition test. European journal of pharmacology. 2013, 701: 27-32.
Johnson, N. J., et al. Trigeminal pathways deliver a low molecular weight drug from the nose to the brain and orofacial structures. Mol. Pharm. 2010, 7: 884-893.
Kozikowski, A. P., et al. Synthesis of urea-based inhibitors as active site probes of glutamate carboxypeptidase II: efficacy as analgesic agents. J. Med. Chem. 2004, 47: 1729-1738.
Lochhead, J. J.; Thorne, R. G. Intranasal delivery of biologics to the central nervous system. Adv. Drug Deliv. Rev. 2011, 64: 614-628.
Long, J. B, et al. Inhibition of glutamate carboxypeptidase II (NAALADase) protects against dynorphin A-induced ischemic spinal cord injury in rats. Eur. J. Pharmacol. 2005, 508: 115-122.
Luszczki, J. J., et al. 2-phosphonomethyl-pentanedioic acid (glutamate carboxypeptidase II inhibitor) increases threshold for electroconvulsions and enhances the antiseizure action of valproate against maximal electroshock-induced seizures in mice. Eur. J Pharmacol. 2006, 531: 66-73.
McKinzie, D. L., et al. NAALADase inhibition reduces alcohol consumption in the alcohol-preferring (P) line of rats. Addiction biology. 2000, 5: 411-416.
Mesters, J. R., et al. Structure of glutamate carboxypeptidase II, a drug target in neuronal damage and prostate cancer. EMBO. J 2006, 25: 1375-1384.
Nagel, J., et al. Effects of NAAG peptidase inhibitor 2-PMPA in model chronic pain-relation to brain concentration. Neuropharmacology 2006, 51: 1163-1171.
Neale, J. H., et al. N-Acetylaspartylglutamate: the most abundant peptide neurotransmitter in the mammalian central nervous system. J. Neurochem. 2000, 75: 443-452.
Neale, J. H. N-acetylaspartylglutamate is an agonist at mGluR(3) in vivo and in vitro. Journal of neurochemistry 2011, 119: 891-895.
Olszewski, R. T., et al. mGluR3 and not mGluR2 receptors mediate the efficacy of NAAG peptidase inhibitor in validated model of schizophrenia. Schizophr. Res. 2012, 136: 160-161.
Olszewski, R. T., et al. NAAG peptidase inhibition reduces locomotor activity and some stereotypes in the PCP model of schizophrenia via group II mGluR. J. Neurochem. 2004, 89: 876-885.
Rahn, K. A., et al. Inhibition of glutamate carboxypeptidase II (GCPII) activity as a treatment for cognitive impairment in multiple sclerosis. Proc. Natl. Acad. Sci. U.S.A. 2012, 109: 20101-20106.
Popik, P., et al. Morphine tolerance and reward but not expression of morphine dependence are inhibited by the selective glutamate carboxypeptidase II (GCP II, NAALADase) inhibitor, 2-PMPA. Neuropsychopharmacology 2003, 28: 457-467.
Rais, R., et al. Bioanalytical method for evaluating the pharmacokinetics of the GCP-II inhibitor 2-phosphonomethyl pentanedioic acid (2-PMPA). Journal of pharmaceutical and biomedical analysis 2014, 88: 162-169.
Rais, R., et al. Reversible disulfide formation of the glutamate carboxypeptidase II inhibitor E2072 results in prolonged systemic exposures in vivo. Drug metabolism and disposition: the biological fate of chemicals 2012, 40: 2315-2323.
Robinson, M. B., et al. Hydrolysis of the brain dipeptide N-acetyl-L-aspartyl-L-glutamate. Identification and characterization of a novel N-acetylated alpha-linked acidic dipeptidase activity from rat brain. The Journal of biological chemistry 1987, 262: 14498-14506.
Rojas, C., et al. Kinetics and inhibition of glutamate carboxypeptidase II using a microplate assay. Analytical biochemistry 2002, 310: 50-54.
Saito, O., et al. Ketamine and N-acetylaspartylglutamate peptidase inhibitor exert analgesia in bone cancer pain. Can. J. Anaesth. 2006, 53: 891-898.
Sasson, N. J., et al. Children with autism demonstrate circumscribed attention during passive viewing of complex social and nonsocial picture arrays. Autism. Res. 2008, 1: 31-42.
Schatz P L, et al. Captopril-induced hypersensitivity lung disease. An immune-complex-mediated phenomenon. Chest. 1989, 95: 685-687.
Shippenberg T S, et al. Modulation of behavioral sensitization to cocaine by NAALADase inhibition. Synapse. 2000, 38: 161-166.
Slusher B S, et al. Selective inhibition of NAALADase, which converts NAAG to glutamate, reduces ischemic brain injury. Nat. Med. 1999, 5:1396-1402.
Stevens J, et al. Systemic and direct nose-to-brain transport pharmacokinetic model for remoxipride after intravenous and intranasal administration. Drug Metab. Dispos. 2011, 39: 2275-2282.
Tortella F C, et al. Neuroprotection produced by the NAALADase inhibitor 2-PMPA in rat cerebellar neurons. Eur. J. Pharmacol. 2000, 402: 31-37.
Van der Post J P, et al. The central nervous system effects, pharmacokinetics and safety of the NAALADase-inhibitor GPI 5693. Br. J. Clin. Pharmacol. 2005, 60: 128-136.
Vaka S R, et al. Delivery of nerve growth factor to brain via intranasal administration and enhancement of brain uptake. J. Pharm. Sci. 2009, 98: 3640-3646.
van Woensel, et al., Formulations for Intranasal Delivery of Pharmacological Agents to Combat Brain Disease: A New Opportunity to Tackle GBM? Cancers 2013, 5: 1020-1048.
Vitharana D, et al. Synthesis and biological evaluation of (R)- and (S)-2-(phosphonomethyl)pentanedioic acids as inhibitors of glutamate carboxypeptidase II. Tet. Asymm. 2002, 13: 1609-1614.
Vornov J J, et al. Pharmacokinetics and pharmacodynamics of the glutamate carboxypeptidase II inhibitor 2-MPPA show prolonged alleviation of neuropathic pain through an indirect mechanism. The Journal of pharmacology and experimental therapeutics 2013, 346: 406-413.
Watkins, J. C. 1-glutamate as a central neurotransmitter: looking back. Biochem. Soc. Trans. 2000, 28: 297-309.
Wozniak K M, et al. The role of glutamate signaling in pain processes and its regulation by GCP II inhibition. Curr. Med. Chem. 2012, 19: 1323-1334.
Witkin J M, et al. NAALADase (GCP II) inhibition prevents cocaine-kindled seizures. Neuropharmacology 2002, 43: 348-356.
Xi Z X, et al. N-acetylaspartylglutamate (NAAG) inhibits intravenous cocaine self-administration and cocaine-enhanced brain-stimulation reward in rats. Neuropharmacology 2010, 58: 304-313.
Xi Z X, et al. Inhibition of NAALADase by 2-PMPA attenuates cocaine-induced relapse in rats: a NAAG-mGluR2/3-mediated mechanism. Journal of neurochemistry 2010, 112: 564-576.
Yamamoto, T., et al. Spinal N-acetyl-alpha-linked acidic dipeptidase (NAALADase) inhibition attenuates mechanical allodynia induced by paw carrageenan injection in the rat. Brain. Res. 2001, 909: 138-144.
Yamamoto T, et al. Inhibition of spinal N-acetylated-alpha linked acidic dipeptidase produces an antinociceptive effect in the rat formalin test. Neuroscience 2001, 102: 473-479.
Yamamoto T, et al. Antinociceptive effects of N-acetylaspartylglutamate (NAAG) peptidase inhibitors ZJ-11, ZJ-17 and ZJ-43 in the rat formalin test and in the rat neuropathic pain model. Eur J Neurosci. 2004, 20: 483-494.
Yamamoto T, et al. Local administration of Nacetylaspartylglutamate (NAAG) peptidase inhibitors is analgesic in peripheral pain in rats. Eur. J. Neurosci. 2007, 25: 147-158.
Yamamoto T, et al. Intracerebroventricular administration of Nacetylaspartylglutamate (NAAG) peptidase inhibitors is analgesic in inflammatory pain. Mol. Pain. 2008, 4: 31.
Zhang W, et al. GCPII (NAALADase) inhibition prevents long-term diabetic neuropathy in type 1 diabetic BB/Wor rats. J. Neurol. Sci. 2002, 194: 21-28.
Zhang W, et al. The preventive and therapeutic effects of GCPII (NAALADase) inhibition on painful and sensory diabetic neuropathy. J. Neurol. Sci. 2006, 247: 217-223.
Zhong C, et al. NAAG peptidase inhibitor reduces acute neuronal degeneration and astrocyte damage following lateral fluid percussion TBI in rats. J. Neurotrauma. 2005, 22: 266-276.
Zhong C, et al. NAAG peptidase inhibitor increases dialysate NAAG and reduces glutamate, aspartate and GABA levels in the dorsal hippocampus following fluid percussion injury in the rat. J. Neurochem. 2006, 97: 1015-1025.

Although the foregoing subject matter has been described in some detail by way of illustration and example for purposes of clarity of understanding, it will be understood by those skilled in the art that certain changes and modifications can be practiced within the scope of the appended claims.

1. A method for delivering a glutamate carboxypeptidase II (GCP-II) inhibitor to a subject, the method comprising administering an effective amount of the GCP-II inhibitor to the subject via an intranasal route.

2. The method of claim 1, wherein the GCP-II inhibitor is selected from the group consisting of a urea-, hydroxamate-, thiol-, and phosphonate-based GCP-II inhibitor.

3. The method of claim 2, wherein the GCP-II inhibitor is selected from the group consisting of (N—[N—[(S)-1,3-dicarboxypropyl] carbamoyl]-cysteine) (DCMC), 2-(3-mercaptopropyl)pentane-dioic acid (2-MPPA), and 2-(phosphonomethyl)-pentanedioic acid (2-PMPA), and stereoisomers and prodrugs thereof.

4. The method of claim 3, wherein the GCP-II inhibitor is 2-(phosphonomethyl)-pentanedioic acid (2-PMPA), and stereoisomers and prodrugs thereof.

5. The method of claim 1, wherein the subject has excess GCP-II activity before the GCP-II inhibitor is administered.

6. The method of claim 5, wherein performing the method results in inhibiting the excess GCP-II activity.

7. The method of claim 1, wherein the method results in an increase in total brain and/or peripheral nervous system concentration and an increase in brain and/or peripheral nervous system-to-plasma partition ratio of the GCP-II inhibitor as compared to using an intraperitoneal route.

8. The method of claim 7, wherein there is an approximately 100-fold or more increase in the brain and/or peripheral nervous system-to-plasma partition ratio as compared to using an intraperitoneal route.

9. The method of claim 1, wherein the GCP-II inhibitor reaches a target organ or system of the subject through an olfactory pathway.

10. The method of claim 9, wherein the target organ or system is a brain and/or peripheral nervous system of the subject.

11. The method of claim 11, wherein performing the method results in almost 100% inhibition of GCP-II enzyme activity in the olfactory bulb and cortex of the brain and at least 70% inhibition in the cerebellum of the brain.

12. A method for treating a neurological disease or disorder in a subject in need of treatment thereof, the method comprising intranasally administering to the subject a therapeutically effective amount of glutamate carboxypeptidase II (GCP-II) inhibitor.

13. The method of claim 12, wherein the GCP-II inhibitor is selected from the group consisting of a urea-, hydroxamate-, thiol-, and phosphonate-based GCP-II inhibitor.

14. The method of claim 13, wherein the GCP-II inhibitor is selected from the group consisting of (N—[N—[(S)-1,3-dicarboxypropyl] carbamoyl]-cysteine) (DCMC), 2-(3-mercaptopropyl)pentane-dioic acid (2-MPPA), and 2-(phosphonomethyl)-pentanedioic acid (2-PMPA), and stereoisomers and prodrugs thereof.

15. The method of claim 14, wherein the GCP-II inhibitor is 2-PMPA, and stereoisomers and prodrugs thereof.

16. The method of claim 12, wherein the method results in an increase in total brain and/or peripheral nervous system concentration and an increase in brain and/or peripheral nervous system-to-plasma partition ratio of the GCP-II inhibitor as compared to using an intraperitoneal route.

17. The method of claim 16, wherein there is an approximately 100-fold or more increase in the brain and/or peripheral nervous system-to-plasma partition ratio using the intranasal route as compared to using an intraperitoneal route.

18. The method of claim 12, wherein the GCP-II inhibitor reaches a target organ or system of the subject through an olfactory pathway.

19. The method of claim 18, wherein the target organ or system a brain and/or peripheral nervous system of the subject.

20. The method of claim 19, wherein performing the method results in almost 100% inhibition of GCP-II enzyme activity in the olfactory bulb and cortex of the brain and at least 70% inhibition in the cerebellum of the brain.

21. The method of claim 12, wherein the neurological disease or disorder is selected from the group consisting of traumatic spinal cord and brain injury, stroke, neuropathic and inflammatory pain, neurological disorder as a result of drug abuse, epilepsy, amyotrophic lateral sclerosis (ALS), schizophrenia, Huntington's disease, neuropathy, multiple sclerosis, cognition impairment, brain cancer, HIV-associated neurocognitive disorder, and cognition impairment associated with neurodegenerative or neuropsychiatric conditions.

22. The method of claim 12, wherein the neurological disease or disorder results in excess GCP-II activity in the brain and/or peripheral nervous system of the subject.

23. The method of claim 22, wherein performing the method results in inhibiting the excess GCP-II activity.

24. A method for diagnosing a neurological disease or disorder involving alteration of glutamate carboxypeptidase II enzyme (GCP-II) levels or location in the brain and/or peripheral nervous system of a subject, the method comprising intranasally administering to the subject an effective amount of GCP-II inhibitor labeled with a fluorescent species or radiolabeled with an isotope and obtaining an image of the brain and/or peripheral nervous system of the subject, wherein an alteration in levels or location of GCP-II in the brain and/or peripheral nervous system as compared to the brain and/or peripheral nervous system of a subject without the neurological disease or disorder is indicative that the subject has the neurological disease or disorder.

25. The method of claim 24, wherein the GCP-II inhibitor is selected from the group consisting of a urea-, hydroxamate-, thiol-, and phosphonate-based GCP-II inhibitor.

26. The method of claim 25, wherein the GCP-II inhibitor is selected from the group consisting of (N—[N—[(S)-1,3-dicarboxypropyl] carbamoyl]-cysteine) (DCMC), 2-(3-mercaptopropyl)pentane-dioic acid (2-MPPA), and 2-(phosphonomethyl)-pentanedioic acid (2-PMPA), and stereoisomers and prodrugs thereof.

27. The method of claim 26, wherein the GCP-II inhibitor is 2-PMPA, and stereoisomers and prodrugs thereof.

28. The method of claim 24, wherein the method results in an increase in total brain and/or peripheral nervous system concentration and an increase in brain and/or peripheral nervous system-to-plasma partition ratio of the GCP-II inhibitor as compared to using an intraperitoneal route.

29. The method of claim 28, wherein there is an approximately 100-fold or more increase in the brain and/or peripheral nervous system-to-plasma partition ratio using the intranasal route as compared to using an intraperitoneal route.

30. The method of claim 24, wherein most of the GCP-II inhibitor reaches the brain and/or peripheral nervous system through the olfactory pathway.

31. The method of claim 24, wherein the neurological disease or disorder is selected from the group consisting of traumatic spinal cord and brain injury, stroke, neuropathic and inflammatory pain, neurological disorder as a result of drug abuse, epilepsy, amyotrophic lateral sclerosis (ALS), schizophrenia, Huntington's disease, neuropathy, multiple sclerosis, cognition impairment, brain cancer, HIV-associated neurocognitive disorder, and cognition impairment associated with neurodegenerative or neuropsychiatric conditions.

32. The method of claim 24, wherein the neurological disease or disorder results in excess GCP-II activity in the brain and/or peripheral nervous system of the subject.

33. The method of claim 24, wherein the isotope is selected from the group consisting of ¹²⁵I, ¹²³I, ¹⁸F, ¹⁴C, and ⁶⁸Ga.

34. A method for imaging glutamate carboxypeptidase II (GCP-II) in a subject, the method comprising intranasally administering to the subject an effective amount of GCP-II inhibitor labeled with a fluorescent species or radiolabeled with an isotope and obtaining an image of the subject.

35. The method of claim 34, wherein the GCP-II inhibitor is selected from the group consisting of a urea-, hydroxamate-, thiol-, and phosphonate-based GCP-II inhibitor.

36. The method of claim 35, wherein the GCP-II inhibitor is selected from the group consisting of (N—[N—[(S)-1,3-dicarboxypropyl] carbamoyl]-cysteine) (DCMC), 2-(3-mercaptopropyl)pentane-dioic acid (2-MPPA) and 2-(phosphonomethyl)-pentanedioic acid (2-PMPA), and stereoisomers and prodrugs thereof.

37. The method of claim 34, wherein the GCP-II inhibitor is 2-(phosphonomethyl)-pentanedioic acid (2-PMPA), and stereoisomers and prodrugs thereof.

38. The method of claim 34, wherein the isotope is selected from the group consisting of ¹²⁵I, ¹²³I, ¹⁸F, ¹⁴C, and ⁶⁸Ga.

39. The method of claim 34, wherein the image of the subject comprises an image of a brain and/or peripheral nervous system of the subject.
